(19) United States
(12) Patent Application Publication
(10) Pub. No.: US 2016/0213396 A1
(43) Pub. Date: Jul. 28, 2016

(54) CATHETER WITH AN AUGER AND METHOD OF USE THEREOF

(71) Applicant: COOK MEDICAL TECHNOLOGIES LLC, BLOOMINGTON, IN (US)

(72) Inventors: Angela R. Dowell, Lafayette, IN (US); Keith R. Milner, West Lafayette, IN (US)

(73) Assignee: COOK MEDICAL TECHNOLOGIES LLC, BLOOMINGTON, IN (US)

(21) Appl. No.: 14/989,349

(22) Filed: Jan. 6, 2016

Related U.S. Application Data
(60) Provisional application No. 62/106,295, filed on Jan. 22, 2015.

(51) Int. Cl. A61B 17/3207

(52) U.S. Cl. CPC A61B 17/320758; A61B 17/3207; A61B 17/320783; A61B 2017/320775; A61B 2017/00991

(57) ABSTRACT

The present invention generally relates to catheters including an auger insert and to methods of using such devices. In certain embodiments, the auger includes an auger blade that is movable between a first position totally within the catheter lumen and a second position extending out of the distal end of the catheter.

### RELATED APPLICATIONS

This patent application claims the benefit of U.S. provisional patent application No. 62/106,295, filed Jan. 22, 2015, the entire contents of which application is hereby incorporated by reference.

### TECHNICAL FIELD

The present invention generally relates a catheter with an auger-type insert and to methods of using such a device. In certain embodiments, the insert may be operated when positioned within the catheter body or, alternatively, when extended beyond the catheter body. In another aspect of the invention, the catheter provides a method for removing a blockage from a body vessel, for example, a vascular vessel.

### BACKGROUND

Various vessels of a human or veterinary subject may be subject to complete or partial blockage for a variety of reasons. For example, arteriosclerosis is a condition where deposits build up along an inner surface of a vascular vessel and cause a partial or total blockage of the vessel. Vascular vessels may also be totally or partially blocked by clotting of blood.

In the case of the vascular system, a variety of treatment methods have been used to either bypass or remove the blockage. For example, coronary by-pass operations provide one method of bypassing a blocked coronary artery. An alternative approach is to open or widen the blocked blood vessel. One technique for reopening a blocked blood vessel is to insert a balloon catheter inside the vessel to expand the vessel and either break loose deposits within the vessel or alternatively, increase the size of the lumen passing through those deposits. In some cases, a stent can be placed at the site of the obstruction to maintain, or even widen, the size of the vessel lumen.

Yet another approach involves the use of a catheter to deliver a fluid, for example a saline solution, under high pressure into the vessel, directly at the site of the obstruction, for example, a blood clot. The fluid jet brakes up the obstruction, remnants of which are removed through the catheter by, for example, applying a vacuum to aspirate the remnants from the vessel and out of the subject's body.

### BRIEF SUMMARY

One aspect of the present invention provides a catheter including a conduit having a proximal end, a distal end, and an internal lumen extending from the proximal end to the distal end. An auger is positioned within the internal lumen. In one embodiment, the conduit includes a fluid connectivity segment having a plurality of holes positioned around a circumference of the conduit and a proximal segment attached to the proximal end of the fluid connectivity segment.

In another embodiment, the auger includes an auger blade that is movable in a proximal-distal direction between a first position, where the auger blade is positioned totally within the internal lumen of the conduit, and a second position wherein the auger blade at least partially extends out of the distal end of the conduit. In yet another embodiment, the auger also includes an elongated core member attached to and extending from a proximal end of the auger blade to the proximal end of the conduit. An auger drive unit may be coupled to the elongated core member, for example, near to or at a proximal end of the elongated core member.

The catheter may also include an end tip and a core wire attached to the proximal end of the end tip. The core wire can be positioned within an auger lumen extending from the distal end of the auger blade to the proximal end of the elongated core member so as to position the proximal end of the end tip adjacent to the distal end of the auger blade.

In some embodiments, the auger blade is rotatable in a clockwise or an anticlockwise direction when viewed from the distal end of the elongated core member. The auger blade can be, for example, a helical auger blade. In certain embodiments, the auger blade is of a size and shape sufficient to create a fluid flow within the lumen directed towards the distal end or the proximal end of the conduit, depending upon the direction of rotation of the auger blade. The auger blade can include a metal, a metal alloy or a polymer or a combination of these materials.

In another embodiment the fluid connectivity segment includes a plurality of woven wires defining the holes and extending distally from attachment points at the distal end of the proximal segment to a proximal end of an end tip. In this embodiment, the fluid connectivity segment can be longitudinally compressible from a fully extended configuration to a shortened configuration.

Another aspect of the present invention provides a method of treating an obstructed vessel. In one embodiment the method includes positioning the distal end of a catheter as disclosed herein at a region within the vessel having the obstruction and rotating the auger blade to create a fluid flow in the lumen directed towards the proximal end of the conduit, where the fluid flow is sufficient to remove material from the obstruction and into the conduit through the holes in the fluid connectivity segment. The catheter may be advanced through the obstruction while rotating the auger blade. In one embodiment, the obstruction is an instruction in a vascular vessel, for example, an artery.

### BRIEF DESCRIPTION OF THE DRAWINGS

FIG. 1 is an illustration showing an exploded view of one embodiment of a distal region of a catheter including an auger.

FIG. 2 is an illustration showing an embodiment of a distal region of a catheter including an auger. Here, the auger is positioned totally within the catheter conduit.

FIG. 3 is an illustration showing another embodiment of a distal region of a catheter including an auger. Here, the auger extends beyond the distal end of the conduit.

FIG. 4 is an illustration showing one embodiment of a distal region of a catheter including an auger. Here, the fluid connectivity segment is formed by a number of woven wires. The fluid connectivity segment is illustrated in a longitudinally extended configuration.

FIG. 5 is an illustration showing the embodiment illustrated in FIG. 4. The fluid connectivity segment is illustrated in a compressed configuration.

### DETAILED DESCRIPTION

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Each disclosed feature or features can be combined with the generalized features discussed herein, to form a disclosed embodiment of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used in the specification, the terms “proximal” and “distal” should be understood as being in the terms of a physician using the device. The term distal means the portion of the device which is farthest from the physician and the term proximal means the portion of the device which is nearest to the physician. The distal portion of the catheter device is that portion of the catheter that is first inserted into the subject while the proximal portion of the catheter generally remains outside the body of the subject.

As used herein, the term “body vessel” means a body passage or lumen, including, but not limited to, vascular coronary and peripheral vessels, esophageal vessels, intestinal vessels, biliary vessels, urethral vessels and ureteral vessels.

One aspect of the present invention provides a catheter including an auger insert. In certain embodiments, the auger may be rotated to create a vortex of fluid flow. When operated within a body vessel of a human or veterinary subject, such fluid flow may allow for or assist in the removal of a partial or total blockage of the body vessel. The body vessel may be, but is not limited to, a vessel of the vascular system, the alimentary system, the urogenital system or the biliary system. In preferred embodiments, the body vessel is a vessel of the coronary or peripheral vascular system.

In one embodiment, the auger is rotated when positioned within a catheter conduit. In this embodiment, the wall of the body vessel is at least partially protected from damage caused by the motion of the auger blade. For example, when the catheter is used to remove a blockage present in a vessel of the vascular system, the body vessel is provided with protection against damage to vascular tissue (for example, the tunica intima, tunica media or venous valves.)

In another embodiment, the auger is rotated while extended at least partially out of the catheter conduit. In this embodiment, the auger blade may be placed closer to the obstruction to allow for increased fluid flow or even in direct contact with the obstruction and can provide for enhanced extraction of a blockage, such as a thrombus or atheroma.

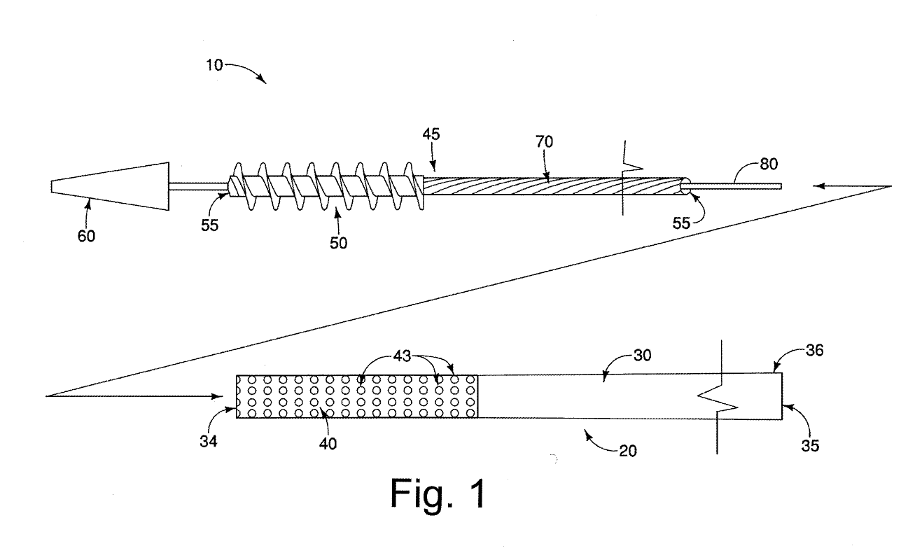

Turning now to FIG. 1, there is illustrated an exploded view of the distal portion of one embodiment of a catheter of the present invention. The distal portion of catheter 10 includes conduit 20 having a proximal end 36, a distal end 34, and an internal lumen 35 extending from the proximal end to the distal end. Auger 45 is sized and shaped to be positioned within the internal lumen.

Conduit 20 includes fluid connectivity segment 40 including a plurality of holes 43 positioned around a circumference of conduit 20 and proximal segment 30 positioned proximally from and connecting to fluid connectivity segment 40. In one embodiment, the plurality of holes extends completely around the circumference of conduit 20. Auger 45 includes auger blade 50 and elongated core member 70 attaching to and extending from the proximal end of auger blade 50. Auger lumen 55 extends from the distal end of auger blade 50 to the proximal end of the elongated core member 70.

In FIG. 1, the proximal end of end tip 60 is shown attaching to core wire 80. When assembled, core wire 80 is contained within auger lumen 55 so as to position the proximal end of end tip 60 adjacent to the distal end of auger blade 50. Auger 45 is contained within the lumen of conduit 20 to position end tip 60 adjacent to distal end 34 of conduit 20. In this configuration, auger blade 50 may be rotated independently of end tip 60. For example, auger blade may be rotated by rotating elongated code member 70 while keeping core wire 80, and end tip 60, from rotating.

In other embodiments, end tip 60 is affixed to the distal end of auger blade 50 such that the two elements always rotate together. In these embodiments, the presence of core wire 80 may not be necessary.

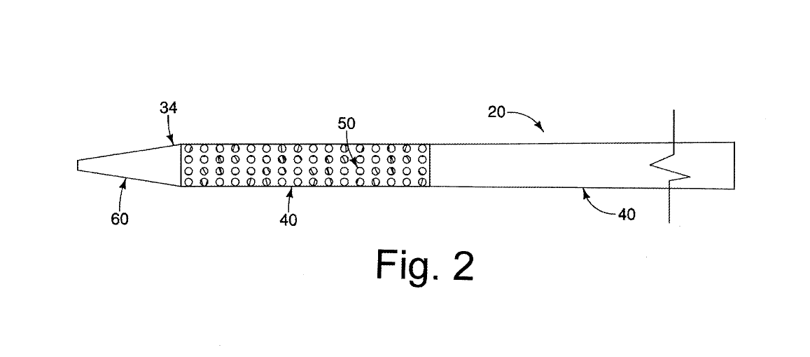

Turning now to FIG. 2. When auger 45 is positioned in a first position such that the proximal end of end tip 60 abuts the distal end 34 of conduit 20, auger blade 50 is positioned within fluid connectivity segment 40 and does not extend distally from fluid connectivity segment 40. When operated with auger blade 50 so positioned, auger blade 50 does not contact the wall of the body vessel, thus protecting the wall from damage due to direct contact with the rotating blade. However, rotating auger blade 50 within conduit 20 will create a vortex and cause fluid flow and therefore act to disrupt any blockage within the vessel. Auger blade 50 is shaped to, depending upon the direction of rotation, either draw in fluid through holes 43 (shown in FIG. 1), or alternatively, to expel fluid from within lumen 35 through holes 43. Auger blade can be driven by an auger drive unit (not shown) which can be connected to the proximal end of elongated core member 70. In one embodiment, such a drive unit is constructed so as to enable the auger blade to be rotated in a clockwise or an anticlockwise direction (when viewed from the distal end of catheter 10.)

When the catheter is used to remove a blockage present in a vessel of the vascular system, auger blade 50 is rotated in a direction causing fluid flow into lumen 35 of conduit 20. Such fluid motion will act to disrupt the obstruction within the body vessel and to deliver debris from the obstruction through holes 43 and into lumen 35 of conduit 20. However, the body vessel is provided with protection against damage as the vessel wall does not directly contact the moving auger blade. Alternatively, auger blade 50 can be rotated in a direction causing fluid flow from lumen 35 of conduit 20 through holes 43. For example, such a mode of operation may be used to deliver a therapeutic agent, such as a thrombolytic, anticoagulant, chelating agent, penetrating agent or permeation aid to the vessel wall. In other embodiments, a fluid, for example saline, may be delivered through conduit 20 to assist in the disruption of the blockage. In one embodiment, the fluid is delivered through a delivery lumen while the auger is operated so as to extract the fluid and material from the blockage.

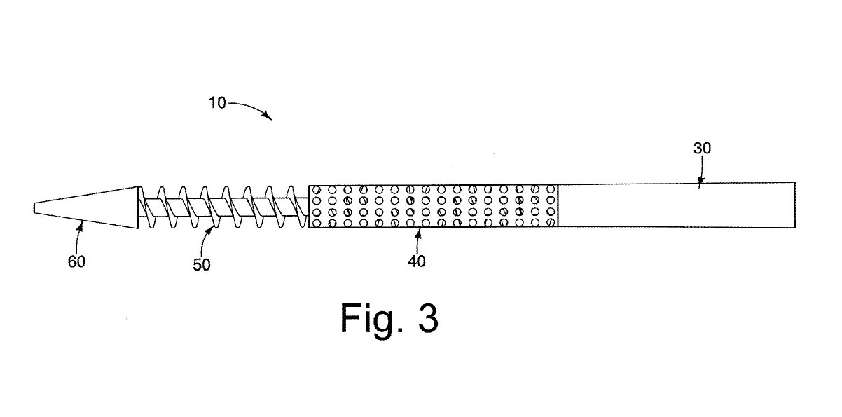

Turning now to FIG. 3, an alternative mode of operation of catheter 10 is illustrated. Here, auger blade 50 is moved from a configuration where auger blade 50 is positioned totally within internal lumen 35 to a second configuration where it extends at least partially out of the distal end of conduit 20. For example, auger 45 may be extended distally with respect to the catheter conduit or the conduit may be retracted proximally to expose at least a portion of the auger. In this latter configuration, auger blade 50 can directly contact the obstruction and may also generate a stronger vortex flow in the region of the obstruction, resulting in increased disruption of the obstruction.

In one embodiment, the auger blade is a helical auger blade, such as auger blade 50. Here, the auger blade consists of a helical blade edge positioned on a central shaft. In alternative embodiments, the auger blade can be, but is not limited to, a helical or serpentine or other shaped wire, or a helical balloon. In some embodiments, the auger blade may not include a central shaft but is instead attached to the distal end of elongated core member 70 by the proximal end of a helical spiral.

The auger blade can include, for example, a metal, a metal alloy or a polymer. Examples of polymers that can be included in the auger blade include, but are not limited to, cellulose acetate, cellulose nitrate, silicone, polyethylene terephthalate, polyurethane, polyamide, polyester (e.g. Nylon), polyorthoester, polyanhydride, polyether sulfone, polycarbonate, polypropylene, high molecular weight polyethylene, and polytetrafluoroethylene, or mixtures of these. Examples of metals ,or metal alloys include, but are not limited to, superelastic nickel-titanium (Ni—Ti) alloys, for example NITINOL, stainless steel, tantalum, titanium, nitinol, cobalt, chromium, nickel, molybdenum, manganese, gold, platinum, inconel, iridium, silver, tungsten, elgiloy and alloys of any of these.

In certain embodiments, the auger blade is an expandable blade. For example, the blade may include a compressible or flexible polymer, or a braided shaped memory metallic construct (for example, NITINOL.) In those embodiments including an expandable auger blade, the blade may be constrained within a conduit having a smaller cross-section but expand to a greater size when moved out of the conduit lumen.

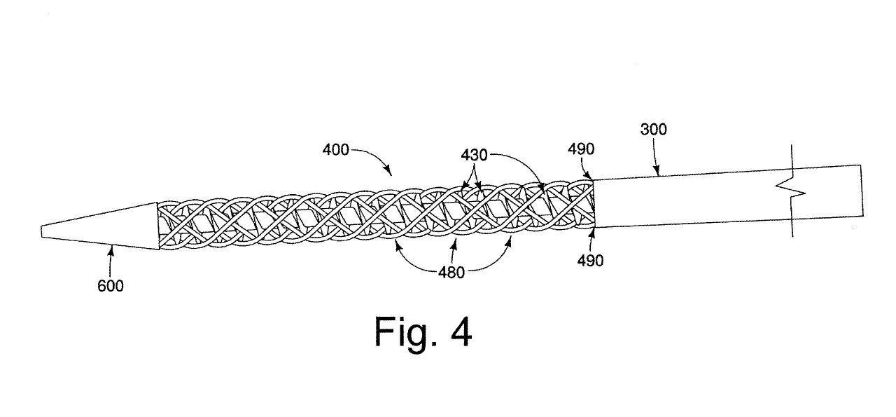

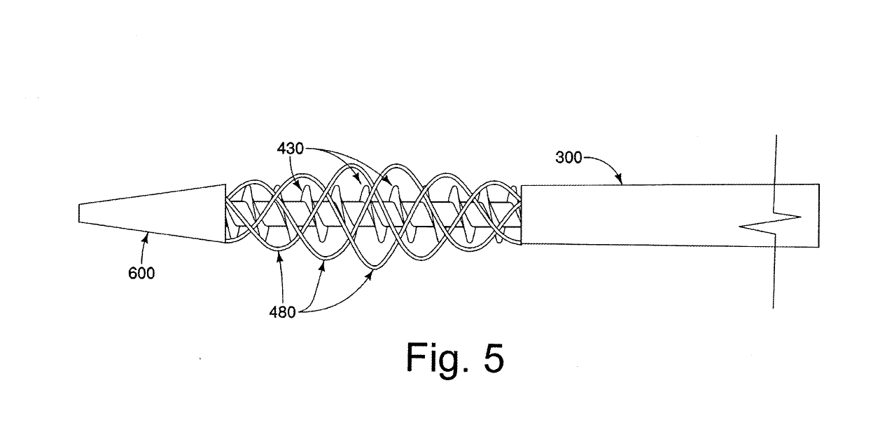

FIGS. 4 and 5 illustrate another embodiment of a catheter of the present invention. In this embodiment, fluid connectivity segment 400 is formed from a plurality of woven wires 480 defining holes 430 in this segment and extending distally from attachment points 490 at the distal end of the proximal segment 300 to attachment points at the proximal end of an end tip 600. In this embodiment, the auger blade is positioned within fluid connectivity segment 400 and an elongated core member extends from the proximal end of the auger blade to the proximal end of the catheter.

As in the embodiment shown in FIGS. 1-3, a core wire may attach to the proximal end of the end tip 600 and extend through an auger lumen and to the proximal end of the catheter. Alternatively, end tip 600 may engage the distal end of the auger blade such that the two elements rotate independently but move longitudinally as a single unit. In this later embodiment, the presence of a core wire may not be required.

In the embodiment shown in FIGS. 4 and 5, fluid connectivity segment 400 is longitudinally compressible from an extended configuration to a compressed (shortened) configuration along a longitudinally (distal-proximal) axis. FIG. 4 illustrates connectivity segment 400 in an extended configuration. This segment may be longitudinally compressed by, for example, moving the core member or core wire in a proximal direction while maintaining the proximal end of the catheter conduit in a stationary position. Alternatively, the core member or the core wire may be maintained in a stationary position while the proximal end of the catheter conduit is moved distally. In those embodiments where the auger blade and the end tip move longitudinally as a single unit, the connectivity segment may be compressed and expanded by moving the auger blade and catheter conduit longitudinally with respect to each other.

FIG. 5 illustrates connectivity segment 400 in a compressed configuration. In this configuration, woven wires 480 move apart, radially resulting in an increase in the size of some of the holes 430 and a lateral expansion of the connectivity segment. For example, the connectivity segment may be expanded enough to the brought into contact with or pass through material of the obstruction. In one embodiment, such a compression allows for an increased fluid vortex to be generated by the auger in the vicinity of the blockage and to further disrupt the blockage, for example, a thrombus or plaque.

In those embodiments including a compressible fluid connectivity segment, the distal end of this segment may be attached to the proximal end of end tip 600. In these embodiments, end tip 600 is constrained by the attachment of woven wires 480 and is therefore not free to rotate along with the auger blade. Alternatively, the distal end of fluid connectivity segment 400 may abut to the proximal end of end tip 600 but not attach to the end tip.

The catheter as disclosed herein can be sized and shaped for delivery to the site of a blockage occurring in a vessel of a human or veterinary subject. For example, the catheter can be sized and shaped for percutaneous delivery to the site of a blockage in the vascular system of the subject. The conduit of a suitable catheter may have an outside diameter of, for example, less than 2.5 millimeters or between 1 and 3 or 2 and 3 or 2 and 4 millimeters and a length of, for example, greater than 200 or 300 or 400 centimeters. The length of the fluid connectivity segment can be, for example, between 5 and 30 or 5 and 20 or 5 and 10 or 10 and 30, or 20 and 30 millimeters.

In those embodiments shown in FIGS. 4 and 5, where fluid connectivity segment 400 is longitudinally compressible from an extended configuration to a compressed configuration along a longitudinally axis, the compressed fluid connectivity segment may extend laterally beyond the lateral dimension of the rest of the catheter conduit and of the exemplary outside diameter dimensions given above. In these configurations, the fluid connectivity segment may be compressed from an extended (fully expanded) longitudinal dimension of, for example, between 40 and 20 mm, 15 and 30 mm, or 20 and 30 mm or 25 and 30 mm or 20 and 25 mm to a compressed configuration of, for example, less than 10 mm, or between 10 and 15 mm, or 10 and 20 mm.

In various embodiments, the holes in the fluid connectivity segment occupy at least 20, 30, 40, 50, 60, 70, 80 or 90 percentage of the total outside surface area of this segment. In determining these figures, the fluid connectivity segment is taken as extending from the distal edge of the most distal hole to the proximal edge of the most proximal hole. In other embodiments, the average largest dimension of the holes is at least 1, 2, 3, 4 or 5 millimeters.

In certain configurations, a guide wire lumen may extend throughout the length of the device. Such a guide wire lumen may be sized to accept a wire guide, for example, a guide wire having a cross sectional dimension of 0.018 or 0.035 inches.

Another aspect of the present invention provides a method of treating a partial or complete obstruction of a body vessel. In one embodiment, the method includes positioning the distal end of a catheter as disclosed herein at a region within the vessel having the obstruction. While in this position, the auger blade is rotated to create a fluid vortex in the vicinity of the obstruction and a fluid flow in the lumen of the catheter conduit directed towards the proximal end of the conduit. The vortex and fluid flow are of a strength sufficient to remove material from the obstruction and deliver this material, through the through the holes in the fluid connectivity segment, to the lumen in the catheter conduit and then to the proximal end of the catheter.

Removal of the obstruction may be performed with the auger blade fully within the fluid connectivity segment. Such a configuration is illustrated in FIG. 2. Alternatively, the obstruction may be removed with the auger blade at least partially extended beyond the distal end of the fluid connectivity segment. This configuration is illustrated in FIG. 3. In this configuration, the auger blade may be brought into physical contact with the obstruction. In other embodiments, the removal of the obstruction may involve the use of the catheter with the auger blade in both of these configurations. When the device includes a compressible fluid connectivity segment that is formed from a plurality of woven wires, removal of the obstruction may be performed fluid connectivity segment in a fully extended configuration or in a compressed configuration.

The method may be utilized to remove an obstruction from a body vessel of a human or veterinary subject. In a preferred embodiment, the vessel is a vascular vessel, such as an artery or a vein. For example, the vascular vessel may be a coronary or peripheral artery. The peripheral artery may be, for example, an artery of the leg, such as the femoral artery.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A catheter comprising:
a conduit comprising a proximal end, a distal end, and an internal lumen extending from the proximal end to the distal end; and
an auger positioned within the internal lumen,
wherein the conduit comprises a fluid connectivity segment comprising a plurality of holes positioned around a circumference of the conduit and a proximal segment, wherein the fluid connectivity segment attaches to a distal end of the proximal segment.

2. The catheter of claim 1, wherein the auger comprises an auger blade,
wherein the auger blade is movable between a first position wherein the auger blade is positioned totally within the internal lumen and a second position wherein the auger blade at least partially extends out of the distal end of the conduit.

3. The catheter of claim 2, wherein the plurality of holes is positioned around the first position.

4. The catheter of claim 2, wherein the auger further comprises
an elongated core member attaching to and extending from a proximal end of the auger blade to the proximal end of the conduit.

5. The catheter of claim 4, further comprising an auger drive unit coupled to the elongated core member.

6. The catheter of claim 4, further comprising an end tip and a core wire,
wherein a distal end of the core wire attaches to a proximal end of the end tip, wherein the auger further comprises an auger lumen extending from a distal end of the auger blade to a proximal end of the elongated core member and wherein the core wire is contained within the auger lumen so as to position the proximal end of the end tip adjacent to the distal end of the auger blade.

7. The catheter of claim 2, wherein the auger blade is a helical auger blade.

8. The catheter of claim 2, wherein the auger blade is rotatable in a clockwise or an anticlockwise direction when viewed from the distal end of the elongated core member.

9. The catheter of claim 2, wherein the auger blade is of a size and shape sufficient to create a fluid flow within the lumen directed towards the distal end or the proximal end of the conduit, depending upon the direction of rotation of the auger blade.

10. The catheter of claim 1, further comprising an end tip,
wherein the fluid connectivity segment comprises a plurality of woven wires defining the holes and extending distally from attachment points at the distal end of the proximal segment to a proximal end of an end tip, wherein the auger comprises an auger blade, positioned within the fluid connectivity segment.

11. The catheter of claim 10, wherein the auger further comprises an elongated core member extending from a proximal end of the auger blade to the proximal end of the conduit.

12. The catheter of claim 11, further comprising a core wire, wherein a distal end of the core wire attaches to a proximal end of the end tip, wherein the auger further comprises an auger lumen extending from a distal end of the auger blade to a proximal end of the elongated core member and wherein the core wire is contained within the auger lumen so as to position the proximal end of the end tip adjacent to the distal end of the auger blade.

13. The catheter of claim 10, wherein the fluid connectivity segment is longitudinally compressible from a fully extended configuration to a shortened configuration.

14. The catheter of claim 13, wherein the fluid connectivity segment is longitudinally compressible from the fully extended configuration by moving the core wire proximally with respect to the conduit.

15. The catheter of claim 14, wherein the auger blade comprises a material selected from the group consisting of a metal, a metal alloy and a polymer.

16. A method of treating an obstructed vessel, the method comprising:
positioning the distal end of a catheter at a region within the vessel having the obstruction, wherein the catheter comprises:
a conduit comprising a proximal end, a distal end, and an internal lumen extending from the proximal end to the distal end; and
an auger positioned within the internal lumen,
wherein the conduit comprises a fluid connectivity segment comprising a plurality of holes positioned around a circumference of the conduit and a proximal segment, wherein the fluid connectivity segment attaches to a distal end of the proximal segment and wherein the auger comprises an auger blade, wherein the auger blade is movable between a first position wherein the auger blade is positioned totally within the internal lumen and a second position wherein the auger blade partially extends out of the distal end of the conduit;
rotating the auger blade while in the first position to create a fluid flow in the lumen directed towards the proximal end of the conduit, wherein the fluid flow is sufficient to remove material from the obstruction through the conduit; and
advancing the catheter through the obstruction while rotating the auger blade while in the first position, whereby material from the obstruction is removed and delivered to the proximal end of the conduit.

17. The method of claim 16, wherein the material from the obstruction enters the lumen through the fluid connectivity segment.

18. The method of claim 16, wherein the auger blade does not directly contact the obstruction.

19. The method of claim 16, wherein the vessel is a vascular vessel.

20. The method of claim 19, wherein the vascular vessel is an artery.
